(19) United States
(12) Patent Application Publication
(10) Pub. No.: US 2008/0078006 A1
(43) Pub. Date: Apr. 3, 2008

(54) Side Shield

(76) Inventor: Fu-Chia Lin, Tainan County (TW)

Correspondence Address: FU-CHIA LIN, 4F, NO. 277, SECTION 3, WUNHUA STREET,, KUEL JEN HSIANG, TAINAN COUNTY 711, TAIWAN, TAIWAN (R.O.C.), omitted

(21) Appl. No.: 11/537,612

(22) Filed: Sep. 30, 2006

(51) Int. Cl. A61F 9/00

(52) U.S. Cl. 2/13

(57) ABSTRACT

A side shield includes a front wall, an interior side wall, and an exterior side wall. The front wall generally overlaps with at least a portion of eyeglasses when in as worn orientation. The exterior side wall extends rearward from the front wall. The interior side wall is attached to the exterior side wall to define a channel between the exterior side wall and the interior side wall. The front wall, the exterior side wall and the interior side wall may be of a unitary construction.

### FIELD OF THE INVENTION

This invention relates generally to eye protection devices, and in particular, to portable side shields.

### BACKGROUND

Eye injuries in the workplace are very common. Common causes for injuries are flying objects, particles, chemicals, dust, and radiant. Eye protection devices have been used to prevent eye injuries. Eye protection devices include non-prescription and prescription safety eyeglasses, goggles, face shields, and side shields. In an area that has flying objects, particles, or dust, it is recommended to wear goggles or safety glasses with side shields.

Goggles are cumbersome to wear on the head and often offer tunnel vision. A person may prefer to wear eyeglasses with side shields. Conventionally, side shields for eyeglasses are designed to be permanently secured to the temple arms of the eyeglasses. Such eyeglasses with permanently attached side shields look strange for casual wear. Removable side shields have thus became more popular.

With removable side shields, a wear can mount side shields on eyeglasses when in hazardous environment and remove the side shields for casual every day wear. However, a problem common with side shields is that the structures are complex and difficult to mount to the eyeglasses. Complex side shield having multiple components complicate manufacture as they require more manufacturing steps and may require more assembly steps to complete a side shield. In addition, such side shields may easily fall apart. Other known portable side shields are designed in a way that side shield is not held security on the eyeglasses. Thus, it would be desirable to provide side shields that reduce or overcomes some or all of the deficiencies inherent in prior known side shields.

### SUMMARY

One aspect of the invention is a side shield having a front wall, an exterior side wall, an interior side wall. The front wall is shaped to generally overlap with at least a portion of eyeglasses. The exterior side wall extending rearward from a portion of the premier of the front wall. The exterior side wall has a nonplaner shape. The interior side wall is integrally attached to the exterior side wall to define a channel between the exterior side wall and the interior side wall. The front wall, the exterior side wall and the interior side wall are of a unitary construction.

Another aspect of the invention is a side shield having an exterior side wall. The exterior side wall has a concave interior surface. An interior side wall extending across the interior surface of the exterior side wall. The interior side wall has two ends. Each end integrally connects to a connecting area of the exterior side wall. Each connecting area is away from a longitudinal line running through a center point of the exterior side wall.

Yet another aspect of the invention is a side shield having a front wall. An exterior side wall extends rearward from the front wall. The exterior side wall has a convex side and a concave side. The interior side wall integrally attaches to the concave side of the exterior side wall to define a channel between the exterior side wall and the interior side wall. The channel has a front opening and a rear opening, the front opening is spaced apart from the front wall.

In some embodiments, the exterior side wall has a non uniform thickness. The exterior side wall is thicker in some areas to provide stiffness when needed. The front opening and the rear opening may be of different sizes. Preferably, the front opening is smaller than the rear opening. When the side shield is attached to the eyeglasses, a temple arm of the side shield insert through the channel created between the exterior and interior side wall. The side shield can be made by simplified manufacturing process, such as injection molding.

The side shield is attached to a temple arm of the eyeglasses. Preferably, the side shield is made of a flexible material by, for example, injection molding.

Substantial advantage is achieved by providing side shield with an attaching structure without sacrificing the benefit of side shields, e.g., flying object protection, and sun or light protection.

These and additional features and advantages disclosed here will be further understood from the following detailed disclosure of certain embodiments.

### BRIEF DESCRIPTION OF THE DRAWINGS

A more complete understanding of the present invention and certain advantages thereof may be acquired by referring to the following detailed description in consideration with the accompanying drawings, in which:

FIG. 1 is a perspective view of an example eyeglasses with side shields in accordance with this invention;

FIG. 2 is a top perspective view of the example side shield in accordance with this invention;

FIG. 3 is a bottom perspective view of the example side shield in accordance with this invention;

FIG. 4 is a cross sectional view of the example side shield, taken along line A-A′ of FIG. 3;

FIG. 5 is a cross sectional view of the example side shield, taken along line B-B′ of FIG. 3;

FIG. 6 is a front view of the example side shield;

FIG. 7 is a side perspective view of the example side shield in accordance with this invention;

FIG. 8 is a bottom perspective view of another example side shield in accordance with this invention; and

FIG. 9 is a front perspective view of exemplary eyeglasses with attached side shield having a temple arm folded.

### DETAILED DESCRIPTION

The present invention may be embodied in various forms. Certain terms used here are considered to be with respect to the wearer of the side shield 10. For example, the term exterior refer to a location away from the head of a wearer of the side shield 10. The term interior refer to a location toward the head of a wearer of side shield 10.

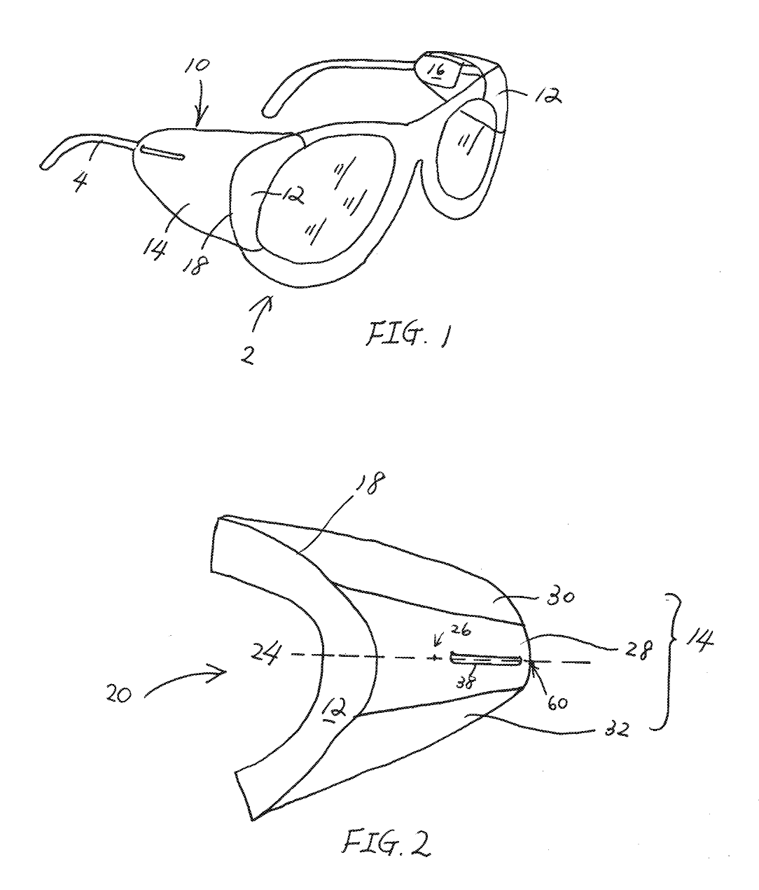

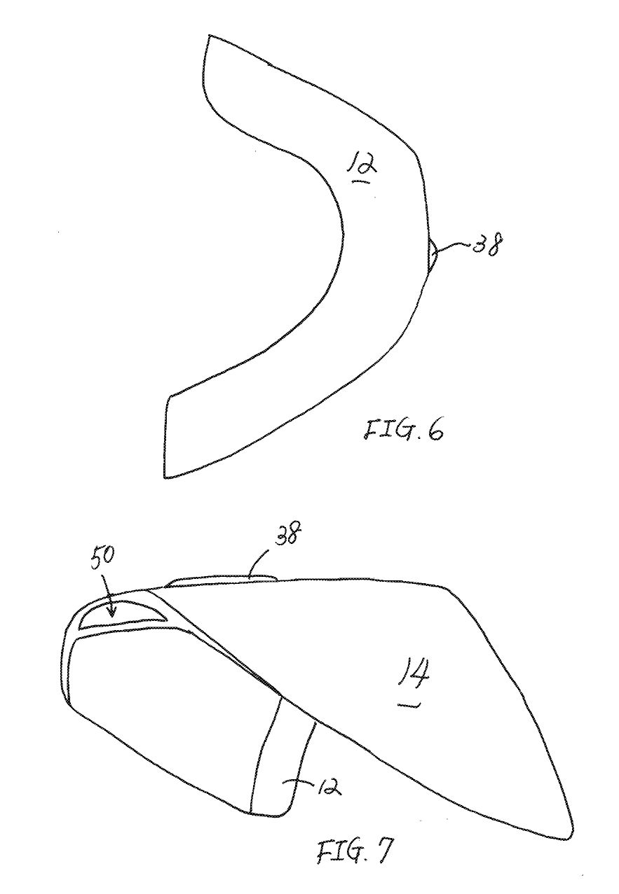

FIG. 1 shows eyeglasses 2 with example side shields 10 in accordance with this invention. In some embodiments, the eyeglasses 2 are prescription eyeglasses, allowing a user to wear their prescriptions while receiving the benefits of side shields 10. The side shield 10 can also be used in sunglasses to block the sun at the lateral edges of the sunglasses. A preferred embodiment of the side shield 10 is shown in more details in FIGS. 2-7. A side shield 10 includes a front wall 12, an exterior side wall 14, and an interior side wall 16 integrally formed as a unitary construction, that is, one-piece, construction with one another. The side shield 10 is preferably formed by injection molding. More preferably, the whole side shield 10 is formed by one single injection molding step, that is, the body of the side shield 10 is formed substantially simultaneously.

The side shield 10 has a flexible structure adopted to the shape of wide range of eyeglasses frames. The side shield 10 is preferred to be formed of an elastomeric or flexible material, for example, a flexible plastic material. An industrial known flexible plastic material, such as flexible polyvinyl chloride (PVC), can be used as a material of the side shield 10. Other suitable material includes flexible thermalplastic rubber (TPR). In some embodiment, the majority of the side shield 10 is formed of an electrometric or flexible material while allowing a small portion of the side shield 10 to be formed of a more rigid material. In other embodiments, the side shield 10 may be tinted to provide Ultra-Violate (UV) protection. The side shield 10 may be formed with additives to provide Infrared Radiation (IR) protection. In a preferred embodiment, the side shield 10 is formed of a clear or translucent material. Formed as such, the side shield 10 does not completely block the peripheral vision of the wearer. Other suitable materials for the side shield 10 will become readily apparent to those skilled in the art, given the benefit of this disclosure.

As shown in FIG. 2, the front wall 12 has a perimeter 18. The exterior side wall 14 extends rearwardly from a portion of the perimeter 18 of the front wall 12. The exterior side wall 14 is non-planar. The exterior side wall 14 has a concave side 20 and a convex side 22, best shown in FIG. 4 and 5. As shown in FIG. 2, the exterior side wall 14 has a longitudinal axis 24 running through a center point 26 of the exterior side wall 14. Exterior side wall 14 includes a central portion 28 extending along the longitudinal axis 24. An upper portion 30 is connected to the central portion 28 and extends upwardly and inwardly from the central portion 28. A lower portion 32 is connected to the central portion 28 and extends downwardly and inwardly from the central portion 28. In some embodiments, the exterior side wall 14 has a general arc shape. The side shield 10 generally encloses a space between the temple aim 4 and the wearer's face. In embodiments where the temple arm 4 is closer to the upper edge of the eyeglasses 2, the lower portion 32 can be larger than the upper portion 30 to better enclose the space between the temple arm 4 and the wearer. As better shown in FIG. 4 and FIG. 5, in the exemplary embodiment, the lower portion 32 is larger than the upper portion 30.

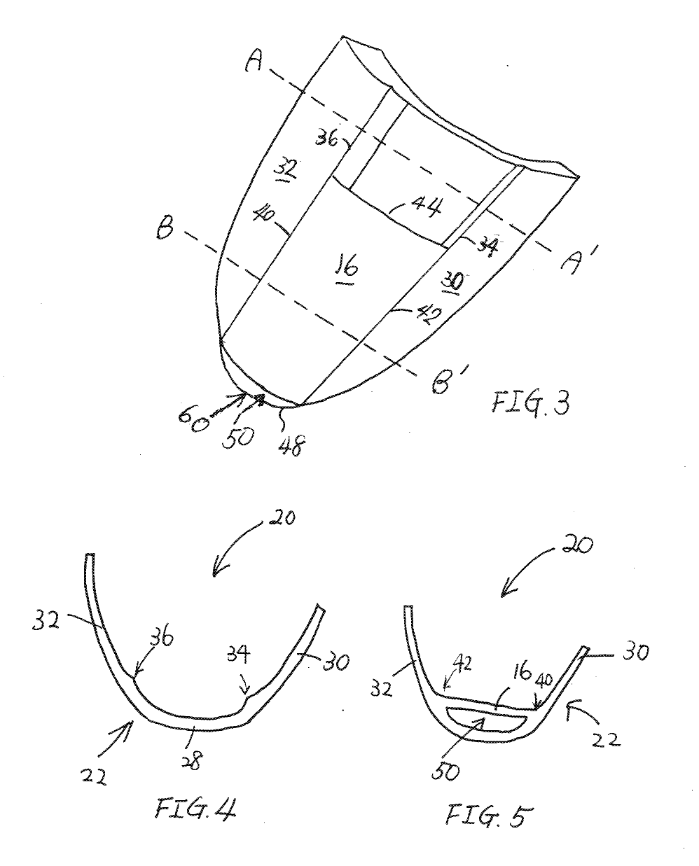

In the illustrated embodiment, the inner surface of the exterior side wall 12 has a step profile. A cross sectional view of the example side shield taken along line A-A′ of FIG. 3 is shown in FIG. 4. The exterior side wall 14 is thicker in the areas close to the connecting lines 34 and 36 between the upper portion 30 and the central portion 28, and between the lower portion 32 and the central portion 28. The thicker areas of the exterior side wall 12 are created to provide stiffness and prevent side shield 10 from pushing into wearer's face through impact. A rib 38 may be formed on the exterior side wall 14, adding stiffness to the exterior side wall 14. The exterior side wall 14 is preferably made of a flexible material and preferably has a non uniform thickness to add stuffiness in some area while being able to flex during use. The rib 38 can be formed by injection molding while forming the other portions of the side shield 10.

FIG. 5 is a cross sectional view of the example side shield, taken along line B-B′ of FIG. 4. As shown in FIGS. 4 and 5, the exterior side wall 14 has the concave side 20 and the convex side 22. The interior side wall 16 extends across the concave side 20 of the exterior side wall 14 to form a bridge. As shown in FIG. 9, the interior side wall 16 is spaced apart from the front wall 12 to expose the temple arm 4 when side shield 10 is attached to the eyeglasses 2. This allows the temple arms 4 to be folded when eyeglasses 2 are not in use. The interior side wall 16 can be a planar or non planar surface. For example, the interior side wall 16 has a radius of curvature larger than that of the central portion 28 of the exterior side wall 14. In a case where the radius of curvature is infinite, the interior side wall 16 is planar. In some embodiments, the central portion 28 of the exterior side wall 14 substantially covers the interior side wall 16 to prevent small flying objects from entering the interior of the side shield 10 from the side whiling allowing utilization of small venting holes (not shown) formed on the exterior side wall 14. Preferably, the apex 60 of the exterior side wall 14 extends further rearwardly than the interior side wall 16 to entirely cover the interior side wall 16 from the side of the wearer.

The interior side wall 14 attached the exterior side wall 14 at two ends 40 and 42. In some embodiment, the end 40 is attached to the connecting line 34 between the central portion 28 and the upper portion 30. The end 42 is attached to the connecting line 36 between the central portion 28 and the lower portion 32. The thickness of the exterior side wall 14 is thicker around the line 40 and 42 to provide stiffness. A channel 50 is formed between exterior side wall 14 and interior side wall 16. In some embodiments, the length of the channel 50 is larger than half length of the exterior side wall 16 for better security. Preferably, the length of the exterior side wall 14 between the front wall 12 and the most rearwaradly point of the exterior side wall 14 is between about 1 inch and about 4 inches and the length of the interior side wall 16 between the front wall 12 and most rearwardly point of the interior side wall 16 is preferably at least 0.5 inches less than the length of the exterior side wall 14 between the front wall 12 and most rearwarly point of the exterior side wall 14. The hinge 56 of the eyeglasses 2 is preferably exposed by the channel 50. This allows the temples arm 4 to be folded even when the side shield 10 is attached to the eyeglasses 2. Advantageously, having the channel 50 on the interior side of the side shield 10 prevents side shields from slipping along the temple arm 4 while folding the temple arm 4.

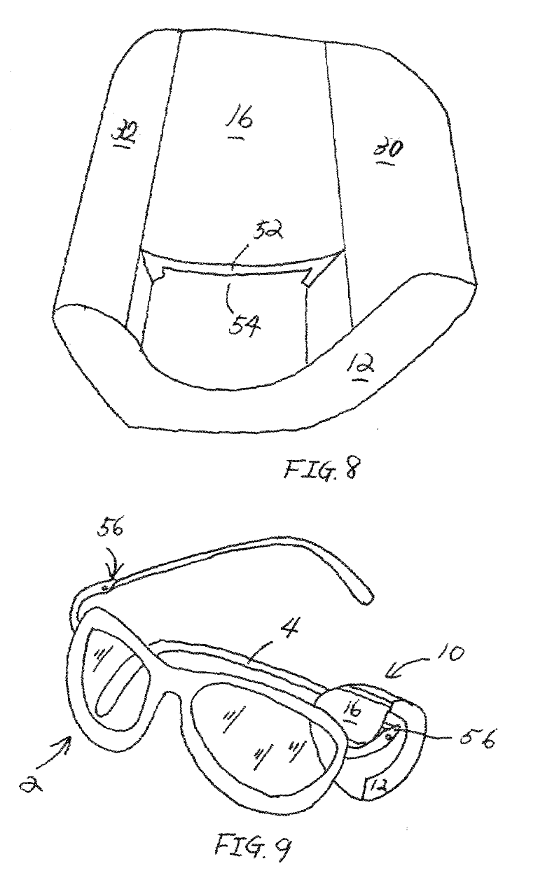

The channel 50 has a front opening 44 and a rear opening 48. To attach the side shield 10 to eyeglasses 2, a user inserts a temple arm 4 of the eyeglasses 2 through front opening 44, the channel 50, and then the rear opening 48. The side shield 10 is then positioned such that front wall 12 is overlapped with the eyeglasses 2 for better protection and to prevent the side shield 10 from slipping off the eyeglasses 2. In the illustrated embodiment, the front opening 44 is spaced apart from the front wall 12 in order to either allow the temple arm 4 being folded or provide more flexibility in the section between the front opening 44 and the front wall 12, or both. In certain embodiments, the front opening 44 of the channel 50 is smaller than the rear opening 48 of the channel 50. Smaller front opening 44 provides better gripping of the temple arm 4. Larger rear opening 48 allows a user to quickly and easily insert or remove the temple arm 4 from the channel 50. The channel 50 need not be equally spaced. Spacing can be varied along the channel 50. For example, the spacing can be smaller in an area closer to the front opening 44 and larger in the area closer to the rear opening 48. In embodiments where the channel 50 is equality spaced, an additional wall 52 with an aperture 54 can be added to the front of the channel 50 to provide smaller front aperture 54 of the channel 50 as shown in FIG. 8. In accordance with this invention, the channel 50 creates an enclosed area for the insertion of a wide range of eyeglasses temple arms. For ease of manufacturing, a slider (not shown) can be inserted into a mold (not shown) to form the channel 50 during injection molding. One advantages of having a channel spacing closer to the rear opening 48 larger is that the removal of the slider is easier. It is appreciated that the manufacture of the side shield 10 is not limited to a single injection molding process. Different portion of the side shield 10 can be injection molded separately and then attach together to form the side shield 10.

In light of the foregoing disclosure of the invention and description of various embodiments, those skilled in this area of technology will readily understand that various modifications and adaptations can be made without departing from the scope and spirit of the invention. All such modifications and adaptations are intended to be covered by the following claims

While the invention has been described in detail in terms of specific examples including presently preferred modes of carrying out the invention, those skilled in the art will appreciate that there are numerous variations and permutations of the above described systems and methods. Thus, the spirit and scope of the invention should be construed broadly as set forth in the appended claims.

We claim:

1. A flexible side shield, comprising:
a front wall shaped to generally overlap with at least a portion of eyeglasses when in as worn orientation, the front wall having a perimeter;
an exterior side wall extending rearwardly from a portion of the perimeter, the exterior side wall having a nonplaner shape;
a interior side wall attaching to the exterior side wall to define a channel between the exterior side wall and the interior side wall; and
the front wall, the exterior side wall and the interior side wall are of a unitary construction.

2. The side shield according to claim 1, wherein the side shield is attached to eyeglasses with a temple arm of the eyeglasses inserted through the channel.

3. The side shield according to claim 1, wherein a material of the side shield include a flexible material.

4. The side shield according to claim 1, wherein a front opening of the channel is smaller than a rear opening of the channel.

5. The side shield according to claim 1, wherein the exterior side wall comprises:
a central portion extending along a longitudinal axis of the exterior side wall;
a upper portion surface extending upwardly from the central portion; and
a lower portion extending downwardly from the central portion.

6. The side shield according to claim 1, wherein a radius of a curvature of the interior side wall is larger than a radius of a curvature of the central portion of the exterior side wall.

7. The side shield according to claim 6, wherein the upper portion is connected to the central portion and extends inwardly.

8. The side shield according to claim 6, wherein the lower portion is connected to the central portion and extends inwardly.

9. The side shield according to claim 6, wherein the upper portion is thicker in an area close to the central portion.

10. The side shield according to claim 6, wherein the lower portion is thicker in an area closer to the central portion.

11. The side shield according to claim 6, further comprising a rib on the central portion of the exterior side wall.

12. The side shield, comprising:
an exterior side wall having a concave inner surface; and
a interior side wall extending across the concave inner surface of the exterior side wall, the interior side wall having two ends, each end integrally connected to a connecting portion of the exterior side wall, the connecting portions of the exterior side wall is thicker than other portions of the exterior side wall.

13. The side shield according to claim 12, wherein a channel is formed between the exterior side wall and the interior side wall for insertion of a temple arm of eyeglasses.

14. The side shield according to claim 13, wherein a front opening of the channel is smaller than a rear opening.

15. The side shield according to claim 13, wherein a length of the channel is larger than half length of the lateral wall.

16. A side shield assembly, comprising:
a front wall;
an exterior side wall extending rearwardly from the front wall, the exterior side wall having a concave side; and
an interior side wall locating on the concave side of the exterior side wall to define a channel between the exterior side wall and the interior side wall, the channel having a front opening and rear opening, wherein the front opening is spaced apart from the front wall.

17. The side shield assembly according to claim 16, further comprising eyeglasses with a temple arm inserted through the channel.

18. The side shield assembly according to claim 16, a length of the interior side wall is preferably at least 0.5 inches less than a length of the exterior side wall.

19. The side shield assembly according to claim 16, wherein the exterior side wall has a non uniform thickness.

20. The side shield assembly to claim 16, wherein the exterior side wall substantially covers the interior side wall.
